(19) United States
(12) Patent Application Publication
(10) Pub. No.: US 2015/0087960 A1
(43) Pub. Date: Mar. 26, 2015

(54) POSITRON EMISSION TOMOGRAPHY GUIDED PROTON THERAPY

(71) Applicant: ProNova Solutions, LLC, Knoxville, TN (US)

(72) Inventor: Jon Treffert, Powell, TN (US)

(21) Appl. No.: 14/492,398

(22) Filed: Sep. 22, 2014

Related U.S. Application Data
(60) Provisional application No. 61/880,559, filed on Sep. 20, 2013.

(51) Int. Cl. A61N 5/10; A61K 51/00; A61B 6/02; A61M 5/00; A61B 6/03; G01R 33/48

(52) U.S. Cl. CPC A61N 5/107; A61B 6/037; G01R 33/481; A61B 6/032; A61N 5/1039; A61N 5/1077; A61B 6/025; A61M 5/007; A61K 51/00; A61N 2005/1087; A61N 2005/1098
USPC 600/411; 600/427; 600/1; 600/4

(57) ABSTRACT

Systems and methods of treating a patient, including a proton treatment system having a proton delivery unit to direct protons to a target area of a patient, the proton treatment system including a positron emission tomography (PET) system having a detector unit to scan for radiotracers introduced into a patient's body, a processing unit to generate location information corresponding to a target area of the patient based on a scanned radiotracer, a guidance unit to receive the location information from the PET system and to instruct the proton delivery unit to direct protons to the target area according to the location information.

### CROSS-REFERENCE TO RELATED APPLICATIONS

This application claims the benefit of U.S. Provisional Patent Application Ser. No. 61/880,559, filed on Sep. 20, 2013, the disclosure of which is incorporated herein in its entirety by reference.

### FIELD OF INVENTION

The present general inventive concept relates to Positron Emission Tomography (PET), and more particularly to utilizing PET to assist proton beam therapy (PT) by dynamic target tracking during radiation treatment.

### BACKGROUND

Radiation used for cancer treatment is called ionizing radiation because it forms ions (electrically charged particles) in the cells of the tissues it passes through. It creates ions by removing electrons from atoms and molecules. This can kill cells or change genes so the cells cannot grow. The ideal radiation with which to treat cancer is one that delivers a defined dose distribution within the target volume and none outside it in order to maximize the dose to the tumor and minimize the dose to surrounding normal tissue.

Ionizing radiation may be sorted into 2 major types: photons (e.g. x-rays and gamma rays), which are most widely used and particle radiation (e.g. electrons, protons, neutrons, carbon ions, alpha particles, and beta particles).

Proton beams (proton beam therapy (PT or PBT)) are an exemplary form of particle beam radiation. Protons are positively charged parts of atoms which cause little damage to tissues they pass through but are very good at killing cells at the end of their path. This means that proton beams may be able to deliver more radiation to the cancer while causing fewer side effects to normal tissues.

For protons and heavier ions, however, the dose increases because the particle penetrates the tissue and loses energy continuously. Hence the dose increases with increasing thickness up to a Bragg peak that occurs near the end of the particle's range. Beyond the Bragg peak, the dose drops to zero (for protons) or almost zero (for heavier ions). The advantage of this energy deposition profile is that less energy is deposited into the healthy tissue surrounding target tissue. Ions are accelerated by means of a cyclotron or synchrotron. The final energy of the emerging particle beam defines the depth of penetration, and hence, the location of the maximum energy deposition. Since it is easy to deflect the beam by means of electro-magnets in a transverse direction, it is possible to employ a raster scan method, i.e., to scan a target area quickly. If the depth of penetration is varied, an entire target volume can be covered in three dimensions, providing an irradiation following the shape of a tumor.

Positron emission tomography (PET) is a nuclear medical imaging technique that produces an image or picture of functional processes in a body. The system detects pairs of gamma rays emitted indirectly by a positron-emitting radionuclide (tracer, radiotracer, radiopharmaceutical, etc.), which is introduced into the body on a biologically active molecule. A radionuclide, or a radioactive nuclide, is an atom with an unstable nucleus, characterized by excess energy available to be imparted either to a newly created radiation particle within the nucleus or via internal conversion. During this process, the radionuclide is said to undergo radioactive decay, resulting in the emission of gamma ray(s) and/or subatomic particles such as alpha or beta particles. These emissions constitute ionizing radiation. Radionuclides are often referred to as radioactive isotopes or radioisotopes.

As the radioisotope undergoes positron emission decay (also known as positive beta decay), it emits a positron, an antiparticle of the electron with opposite charge. The emitted positron travels in tissue for a short distance (typically less than 1 mm, dependent on the isotope), during which time it loses kinetic energy, until it decelerates to a point where it can interact with an electron. The encounter annihilates both electron and positron, producing a pair of annihilation (gamma) photons moving in approximately opposite directions. These are detected when they reach a scintillator in a scanning device, creating a burst of light which is detected by photomultiplier tubes, silicon avalanche photodiodes (Si APD), or silicon photomultipliers (Si PM).

Three-dimensional distribution of radionuclide concentration within the body may be constructed by computer analysis in the PET process.

Efforts regarding positron emission tomography and proton therapy have led to continuing developments to improve their versatility, practicality and efficiency.

### BRIEF SUMMARY

Example embodiments of the present general inventive concept can be achieved by providing a proton delivery guidance system for use with a proton treatment system, the proton treatment system having a proton delivery nozzle to direct protons to a target area of a patient, the proton delivery guidance system including a positron emission tomography (PET) system having a detector unit to scan for radiotracers introduced into a patient's body, the PET system including a processing unit to generate location information of an image corresponding to a target area of the patient, and a guidance unit to receive the location information from the PET system and to instruct the proton treatment system to direct protons to the target area according to the location information.

The detector unit can include a partial ring-shape having an opening therein, and the guidance unit can include a motion control unit configured to control movement of the detector unit such that the proton delivery nozzle directs protons to the target area through the opening while the detector unit scans for radiotracers in the patient's body.

The PET system can be a combined PET/computed tomography (CT) or PET/magnetic resonance imaging (MRI) system.

The detector unit can be a time-of-flight capable detector unit, and the processing unit can utilize limited angle tomographic reconstruction to compensate for incomplete sampling and to estimate radiotracer distribution within the patient's body.

The proton treatment system can include a gantry wheel to rotate the proton treatment nozzle around the patient, and the motion control unit can be configured to control rotation of the gantry wheel according to the location information.

Example embodiments of the present general inventive concept can also be achieved by providing a proton therapy (PT) treatment system, including a positron emission tomography (PET) system to scan for radiotracers in a patient, a processor to determine concentrations of the radiotracers in a target area of the patient and to provide radiotracer location data, a proton beam delivery unit to direct protons to the target area, and a guidance system to control the proton beam delivery unit to direct protons to the target area utilizing the radiotracer location data.

The PET system can scan for radiotracers simultaneously while the proton beam delivery unit directs protons to the targeted area. This can be done in real-time.

The processor can utilize a dynamic tumor tracking algorithm to provide the radiotracer location data.

The proton beam delivery unit can be configured to direct protons of different energies with different Bragg peaks at different depths to the target area.

Example embodiments of the present general inventive concept can also be achieved by providing a method of treating a patient using proton beam therapy (PT), including injecting a patient with one or more radiotracers, scanning for at least one of the radiotracers utilizing positron emission tomography (PET), locating concentrations of the at least one radiotracer in a target area of a patient, generating radiotracer location data of the target area, and radiating the patient with proton beam therapy (PT) utilizing the radiotracer location data, wherein the locating and radiating operations are performed simultaneously in real-time.

The locating operation may include utilizing a dynamic tumor tracking algorithm.

Protons of different energies with different Bragg peaks at different depths can be applied in the PT.

The PET can utilize a compound labeled with a positron emitting radionuclide which localizes in the target tumor, such as [18F] flourodeoxyglucose.

Example embodiments of the present general inventive concept can also be achieved by providing a proton treatment system having a proton delivery unit to direct protons to a target area of a patient, the proton treatment system including a positron emission tomography (PET) system having a detector unit to scan for radiotracers introduced into a patient's body, a processing unit to generate location information corresponding to a target area of the patient based on a scanned radiotracer, and a guidance unit to receive the location information from the PET system and to instruct the proton delivery unit to direct protons to the target area according to the location information.

The detector unit can include a partial ring-shape having an opening therein. The proton delivery unit can include a gantry wheel to rotate a proton delivery nozzle around the patient. The guidance unit can include a motion control unit to control movement of the detector unit and the gantry wheel such that the proton delivery nozzle directs protons to the target area through the opening while the detector unit scans for radiotracers in the patient's body.

Additional features and embodiments of the present general inventive concept will be set forth in part in the description which follows, and may be obvious from the description, or may be learned by practice of the present general inventive concept.

### BRIEF DESCRIPTION OF THE FIGURES

The following example embodiments are representative of example techniques and structures designed to carry out objectives of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 is a graphic schematic side view diagram of a PT and PET combination system according to an example embodiment of the present general inventive concept;

FIG. 2 is a graphic schematic front view diagram of a PT and PET combination system according to an example embodiment of the present general inventive concept;

FIG. 3 is a magnified schematic front view of a portion of a PT and PET combination system according to an example embodiment of the present general inventive concept;

FIG. 4 is a flow diagram of illustrating operations of PET guided PT according to an example embodiment of the present general inventive concept; and

FIG. 5 is a schematic diagram illustrating a proton therapy system configured in accordance with an example embodiment of the present general inventive concept.

### DETAILED DESCRIPTION

Reference will now be made to the example embodiments of the present general inventive concept, examples of which are illustrated in the accompanying drawings and illustrations. The example embodiments are described herein in order to explain the present general concept by referring to the figures.

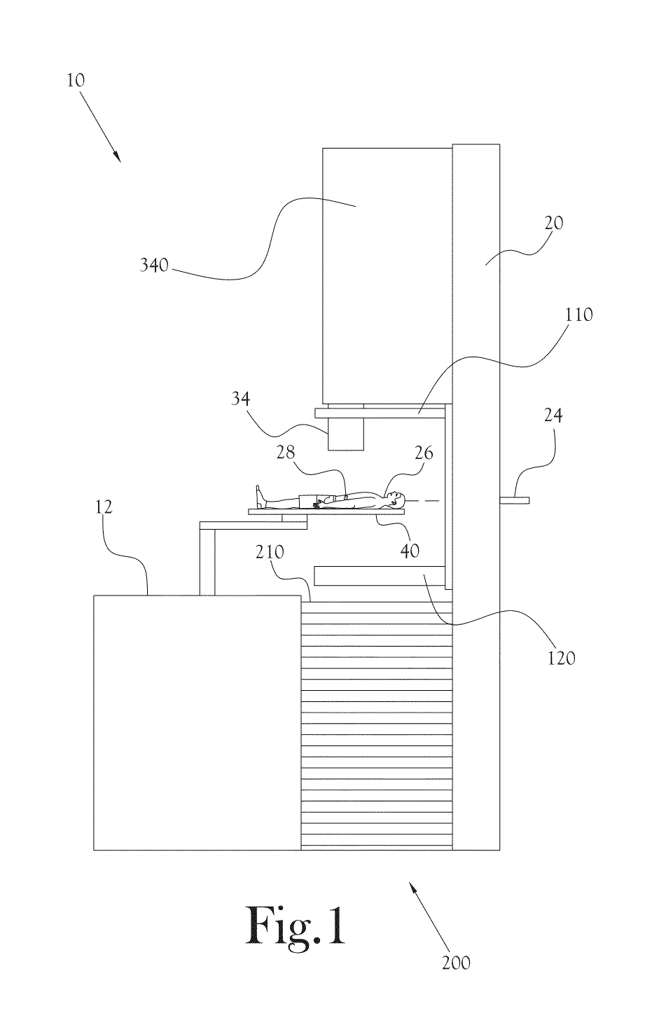

FIG. 1 illustrates an example embodiment of a proton therapy, or proton treatment (PT) system 10 wherein a gantry wheel 20 rotates a proton beam generator nozzle 34 about an axis of rotation 24. A proton beam generator (generally referred to by reference number 340) directs a proton beam through a nozzle 34 from any angle between zero and 380 degrees toward a patient 26 lying on a bed 40 near the isocenter 28 of the gantry wheel which corresponds to a treatment region of a patient. In addition to the proton beam generator 340, a positron emission tomography (PET) system 110, 120, is provided on the gantry system. The PET system 110, 120 can take a variety of shapes, sizes, and configurations. By way of example, but not by way of limitation, the PET system can include two or more flat panels 110, 120 as illustrated in FIG. 1. It is understood that various other PET configurations, such as a partial ring or curved panels, could also be used, separately or in combination with flat panels, without departing from the broader scope of the present general inventive concept.

The gantry system 10 may include a mezzanine platform 12 support system and moving (or rolling) floor 210 for a technician or operator to walk on, enabling access to a patient, magnets, nozzles, achromat, hoses from a beamline, cooling system, etc. for service or replacement. The moving floor may be supported by a moving floor system 200.

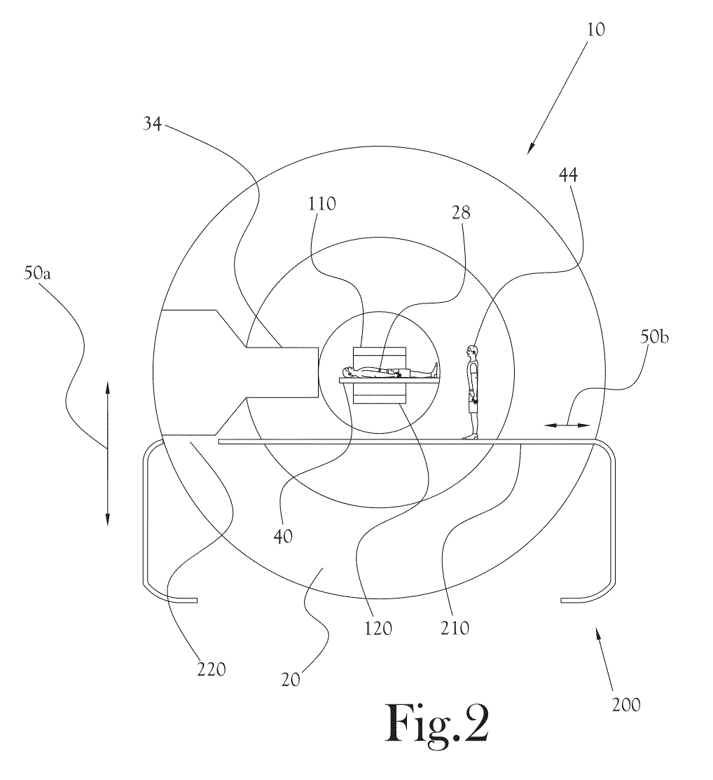

FIG. 2 illustrates an example embodiment of a proton therapy (PT) system 10 wherein a gantry wheel 20 rotates a proton beam generator nozzle 34 about an axis of rotation 24. A proton beam generator directs a proton beam through a nozzle 34 from any angle between zero and 380 degrees toward a patient 26 lying on bed 40 near the isocenter 28 of the gantry wheel which corresponds to a treatment region of a patient. In addition to the proton beam generator, a positron emission tomography (PET) system 110, 120, is provided as part of the gantry system. The gantry system 10 includes a proton beam nozzle apparatus 34 mounted on and rotated by a gantry 20 from a neutral or 0° angle illustrated in FIG. 2 through 380° (position not shown).

An example embodiment moving floor system 200 provides a moving platform 210 on which an operator 44 may stand on, the floor moving in directions indicated by arrows 50a, 50b. The moving floor 210 may have an opening 220 provided therein for providing clearance for the proton beam nozzle apparatus 34 when the beam nozzle is rotated underneath the patient below the floor 220. As the beam nozzle 34 rotates around the patient, it is possible to provide the opening 220 to allow the nozzle 34 to protrude through the opening when at least a portion of the nozzle is rotated below the floor.

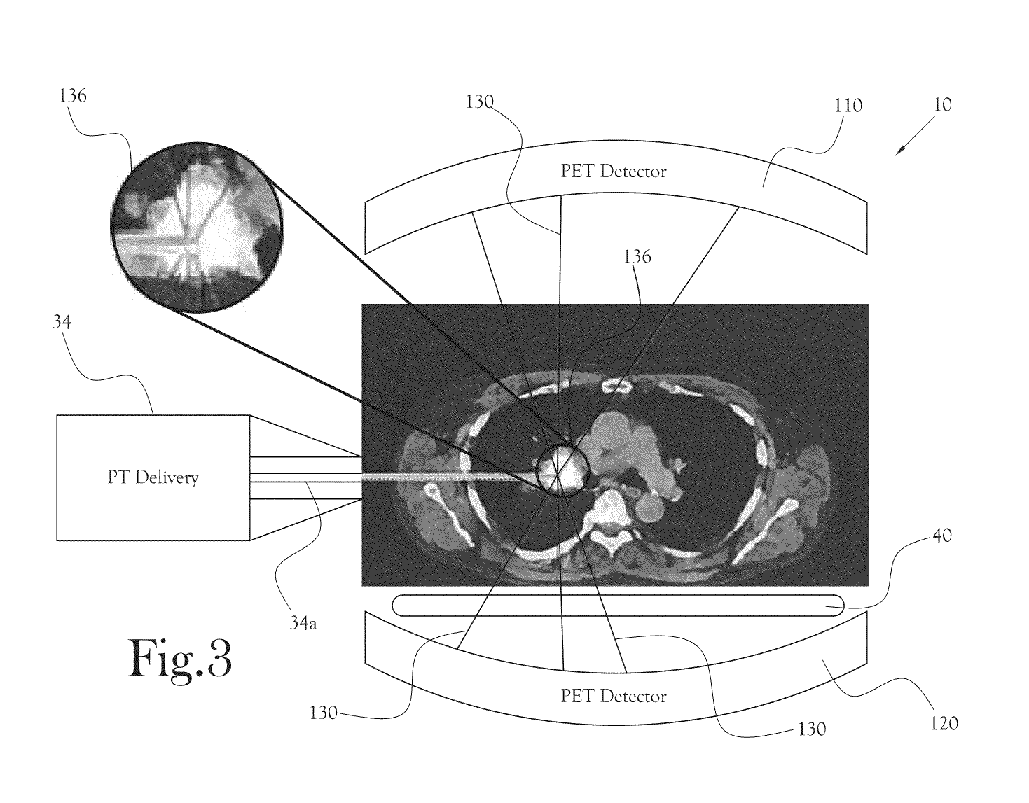

FIG. 3 illustrates an example embodiment of a proton therapy (PT) system 10 wherein a proton beam 34a is directed through the nozzle 34 toward a targeted region 136 of a patient, as illustrated in the PET scan photo of FIG. 3, which can be visible to the patient and/or operator during proton delivery via a display screen. The targeted region 136 is typically located near the isocenter 28 of the gantry wheel 20 (see FIG. 1). A positron emission tomography (PET) system 110, 120 can be provided as part of the gantry wheel 20 or as a separate unit. The PET system can take a variety of shapes and sizes. The PET system can be formed as a partial ring. PET imaging and proton delivery can occur simultaneously in real time. The PET system and proton nozzle can move independently of one another as separate units, or they can move in unison as connected parts to the gantry wheel 20. The PET system is utilized to provide or obtain information or data on the location of the treatment region. The proton beam generator uses the location data to treat the treatment region with particle ionizing radiation at any angle toward the treatment area 136. Location data acquisition is represented by lines 130.

In an example embodiment, the PET may be utilized to produce tomographic images of specific areas of the body. A partial ring detector geometry can allow for the acquisition of data during proton beam delivery. Time of flight (TOF) capable detectors and limited angle tomographic reconstruction techniques can be used to compensate for incomplete sampling and for estimation of the three-dimensional radionuclide distribution within the body. Daily volumetric (e.g. cone-beam CT) x-ray imaging information, planning CT images and structures identified during treatment planning are incorporated in the data processing to identify PET data associated with the target tumor volume (and compensate for attenuation of PET within the body).

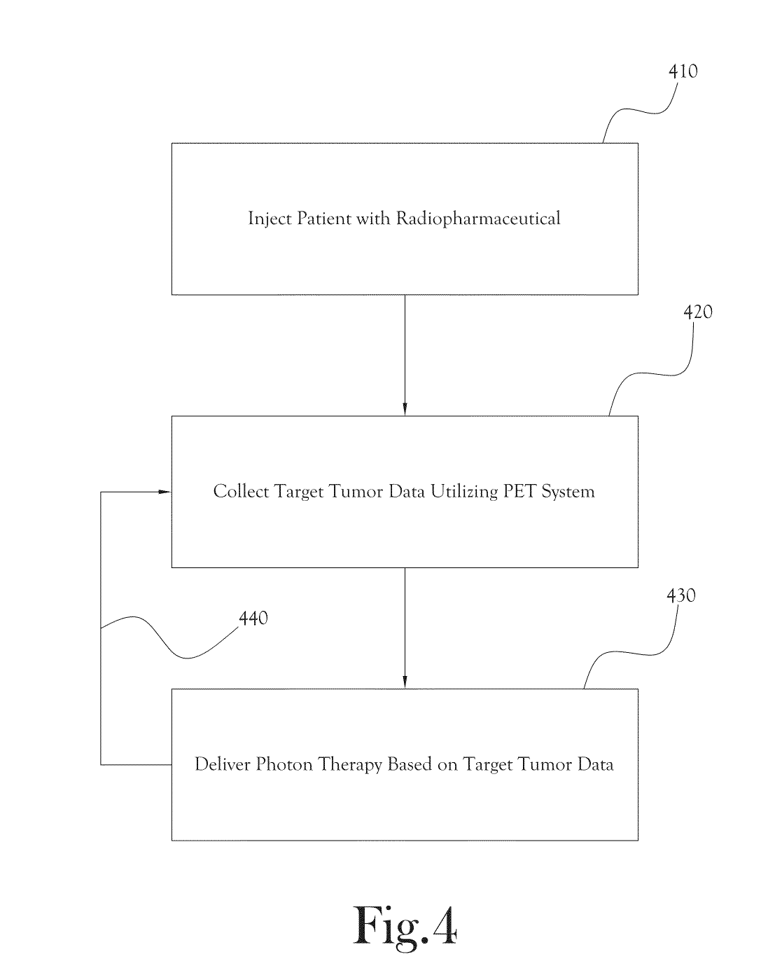

Referring to FIG. 4, prior to proton therapy treatment, a patient is injected with a PET radiopharmaceutical (radioisotope) in a step 410, which localizes preferentially in an active tumor, including a target tumor. PET emission data is collected intra-treatment in a step 420. Utilizing a treatment plan, PET data may be processed in real-time to determine: target (tumor) position/location; spatial position; distribution, etc. PT delivery may be adapted to changes in tumor position or distribution relative to the data collected and a treatment plan. PET data may be obtained dynamically or intra-treatment 440.

In an example embodiment, a processor programmed PET dynamic tumor tracking algorithm may be utilized to estimate target position information utilizing the tumor center of mass (CoM) of segmented target volume on gated PET images which are continuously updated throughout a scan.

In an example embodiment, the PET nuclear medical imaging technique produces a three-dimensional image or picture of the body by detecting pairs of gamma rays emitted indirectly by a positron-emitting radionuclide (tracer) introduced into the body on a biologically active molecule. Three-dimensional images of tracer concentration within the body may then be constructed by computer or processor analysis.

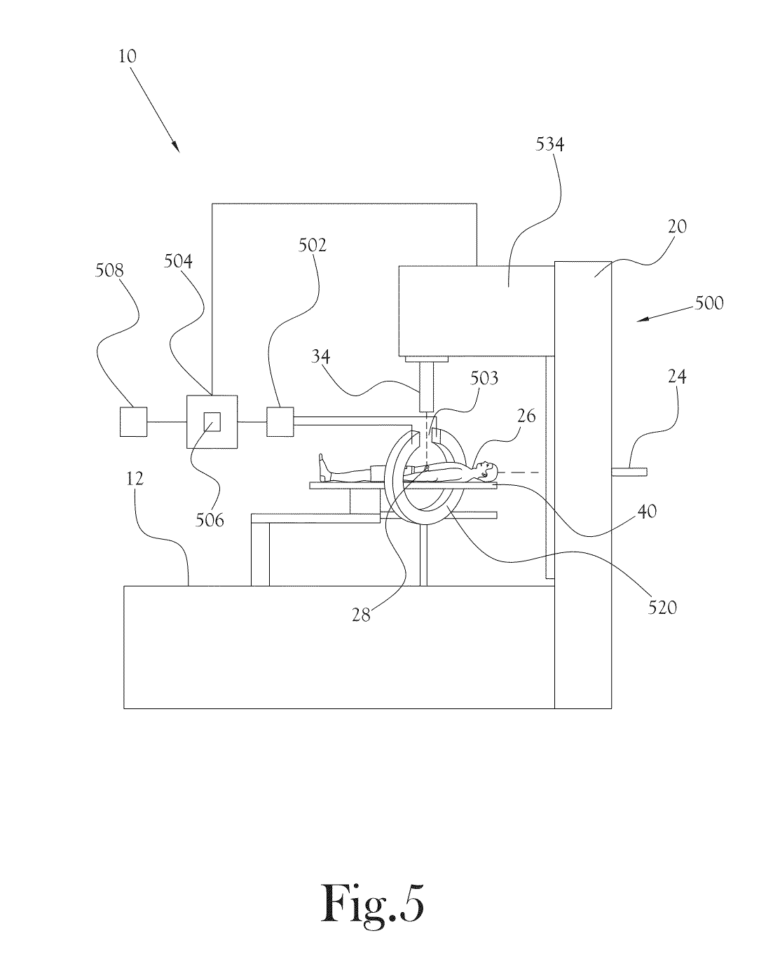

FIG. 5 is a schematic diagram illustrating a proton therapy system configured in accordance with an example embodiment of the present general inventive concept. FIG. 5 illustrates a proton treatment system 500 including a proton delivery system 534 having a proton delivery nozzle 34 to direct protons from a proton beam generator (not shown in FIG. 5) to a target area 28 of a patient 26. The proton treatment system 500 may include a gantry wheel 20 to rotate the proton delivery nozzle around a patient lying on a patient bed 40. As illustrated in FIG. 5, the proton treatment system can include a PET detector unit 520 to detect radiotracers introduced into the patient, especially around the treatment area 28. The detector unit can take the form of a partial ring with an opening 503 therein to enable the proton delivery nozzle 34 to deliver protons to the patient while the detector unit is scanning the patient for radiotracers.

Referring to FIG. 5, the detector unit is connected to a processor 502 which is configured to process the PET data for image reconstruction and location information corresponding to the target area, for example using PET coincidence processing. The processor 502 includes various electronic, optical, and/or solid state componentry configured to receive and utilize x-ray imaging information, such as CT and/or MRI data obtained during treatment planning to register the PET image and location data associated with the target area. The processor 502 can be connected to a guidance unit 504 to control the proton delivery unit 534 to direct protons to the target area according to the location information. The guidance unit can include various electronic and/or electromechanical componentry configured to generate and send control signals (e.g., binary switching signals) to electronic/solid state componentry of the proton delivery unit 534 to enable the proton delivery unit to direct protons to the patient while the detector unit is scanning the patient. The guidance unit 504 can include a motion controller 506 (e.g., robotic articulating members) to control movement of the detector unit 520 and/or proton delivery system 534 and gantry wheel 20 such that the proton delivery nozzle directs protons to the target area through the opening while the detector unit scans for radiotracers in the patient's body. A display unit 508 can be provided to receive and display images and/or location information of the treatment area to the patient and/or operator during proton treatment.

In an example embodiment, three dimensional imaging may be accomplished with the aid of a CT X-ray scan or magnetic resonance imaging (MRI) scan performed on the patient during the same session, in the same machine.

In an example embodiment, the biologically active molecule chosen for the PET is fluorodeoxyglucose (FDG), wherein the concentrations of radiotracer imaged will indicate tissue metabolic activity by virtue of regional glucose uptake. Other radiotracers may be used in the PET to image the tissue concentration of other types of molecules of interest.

In an example embodiment, the PET system utilizes a [18F] labeled radiotracer.

In an example embodiment, protons of different energies with Bragg peaks at different depths are applied in the PT process.

In an example embodiment, a method of treating a patient includes: injecting a patient with radiotracers; scanning for the radiotracers utilizing positron emission tomography (PET); locating concentrations of the radiotracers in a target area and providing radiotracer location data; radiating the patient with proton beam therapy (PT) utilizing the radiotracer location data, wherein the locating and radiating are performed in real-time. The method may include utilizing a dynamic tumor tracking algorithm. The method may include utilizing protons of different energies with Bragg peaks at different depths are applied in the PT.

In an example embodiment, a system for treating a patient includes: a positron emission tomography (PET) for scanning for radiotracers in the patient; a processor for determining concentrations of the radiotracers in a target area and providing radiotracer location data; a proton beam therapy (PT) system for radiating the patient utilizing the radiotracer location data; wherein the determining and radiating are performed in real-time. The system may include utilizing a dynamic tumor tracking algorithm. The system may include utilizing protons of different energies with Bragg peaks at different depths are applied in the radiating.

While the present general inventive concept has been described in relation to certain example embodiments in detail, it is not the intention of the applicant to restrict or in any way limit the scope of the appended claims to such detail. Additional modifications may readily appear to those skilled in the art. The claimed subject matter in its broader aspects is therefore not limited to the specific details, representative apparatus and methods, and illustrative examples shown and described. Accordingly, departures may be made from such details without departing from the spirit or scope of applicant's general concept.

1. A proton delivery guidance system for use with a proton treatment system, the proton treatment system having a proton delivery nozzle to direct protons to a target area of a patient, the proton delivery guidance system comprising:
a positron emission tomography (PET) system having a detector unit to scan for radiotracers introduced into a patient's body, the PET system including a processing unit to generate location information of an image corresponding to a target area of the patient; and
a guidance unit to receive the location information from the PET system and to instruct the proton treatment system to direct protons to the target area according to the location information.

2. The proton delivery guidance system of claim 1, wherein the detector unit comprises a partial ring-shape having an opening therein, and the guidance unit includes a motion control unit configured to control movement of the detector unit such that the proton delivery nozzle directs protons to the target area through the opening while the detector unit scans for radiotracers in the patient's body.

3. The proton delivery guidance system of claim 1, wherein the PET system is a combined PET/computed tomography (CT) or PET/magnetic resonance imaging (MRI) system.

4. The proton delivery guidance system of claim 1, wherein the detector unit is a time-of-flight detector unit, and the processing unit utilizes limited angle tomographic reconstruction to compensate for incomplete sampling and to estimate radiotracer distribution within the patient's body.

5. The proton delivery guidance system of claim 2, wherein the proton treatment system includes a gantry wheel to rotate the proton treatment nozzle around the patient, and the motion control unit is configured to control rotation of the gantry wheel according to the location information.

6. A proton therapy (PT) treatment system, comprising:
a positron emission tomography (PET) system to scan for radiotracers in a patient;
a processor to determine concentrations of the radiotracers in a target area of the patient and to provide radiotracer location data;
a proton beam delivery unit to direct protons to the target area; and
a guidance system to control the proton beam delivery unit to direct protons to the target area utilizing the radiotracer location data.

7. The system of claim 6, wherein the PET system scans for radiotracers and the proton beam delivery unit directs protons to the targeted area simultaneously in real-time.

8. The system of claim 6, wherein the processor utilizes a dynamic tumor tracking algorithm to provide the radiotracer location data.

9. The system of claim 6, wherein the proton beam delivery unit is configured to direct protons of different energies with different Bragg peaks at different depths to the target area.

10. A method of treating a patient using proton beam therapy (PT), comprising:
injecting a patient with one or more radiotracers; scanning for at least one of the radiotracers utilizing positron emission tomography (PET);
locating concentrations of the at least one radiotracer in a target area of a patient;
generating radiotracer location data of the target area; and
radiating the patient with proton beam therapy (PT) utilizing the radiotracer location data,
wherein the locating and radiating operations are performed simultaneously in real-time.

11. The method of claim 10, wherein the locating comprises utilizing a dynamic tumor tracking algorithm.

12. The method of claim 10, wherein protons of different energies with different Bragg peaks at different depths are applied in the PT.

13. The method of claim 10, wherein the PET utilizes a compound labeled with a positron emitting radionuclide which localizes in the target tumor.

14. A proton treatment system having a proton delivery unit to direct protons to a target area of a patient, the proton treatment system comprising:
a positron emission tomography (PET) system having a detector unit to scan for radiotracers introduced into a patient's body;
a processing unit to generate location information corresponding to a target area of the patient based on a scanned radiotracer; and
a guidance unit to receive the location information from the PET system and to instruct the proton delivery unit to direct protons to the target area according to the location information.

15. The proton treatment system of claim 14, wherein the detector unit comprises a partial ring-shape having an opening therein, the proton delivery unit including a gantry wheel to rotate a proton delivery nozzle around the patient, and the guidance unit including a motion control unit to control movement of the detector unit and the gantry wheel such that the proton delivery nozzle directs protons to the target area through the opening while the detector unit scans for radiotracers in the patient's body.
